(19) United States
(12) Patent Application Publication
(10) Pub. No.: US 2008/0228199 A1
(43) Pub. Date: Sep. 18, 2008

(54) ENDOSCOPIC TISSUE APPROXIMATION METHOD

(75) Inventors: Michael S. Cropper, Edgewood, KY (US); Michael J. Andreyko, Cincinnati, OH (US); Richard F. Schwemberger, Cincinnati, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Ethicon Endo-Surgery, Inc., Cincinnati, OH (US)

(21) Appl. No.: 11/687,477

(22) Filed: Mar. 16, 2007

(51) Int. Cl. A61B 17/128

(52) U.S. Cl. 606/139

(57) ABSTRACT

The present invention generally provides methods and devices for approximating tissue. The methods and devices utilize a device for applying an implantable tissue fastener and a variety of implantable tissue fasteners. The tissue-fastening device can be delivered endoscopically and can be adapted to function along side or in conjunction with a flexible endoscope. In general, the device can include a flexible shaft having an implantable tissue fastener applier disposed at a distal end thereof and a handle for operating the implantable tissue fastener applier disposed at a proximal end thereof. A variety of implantable tissue fasteners can be used with the tissue fastener applier device including single and multi-anchor embodiments.

### FIELD OF THE INVENTION

The present invention relates to methods and devices for approximating tissue.

### BACKGROUND OF THE INVENTION

Gastroesophageal reflux disease (GERD) is a common upper gastrointestinal disorder. GERD is a condition in which acidic contents of the stomach flow inappropriately from the stomach into the esophagus. Chronic irritation of the esophagus leads to inflammation of the esophagus, known as esophagitis. In addition to esophagitis, complications of GERD include Barrett's esophagus, esophageal stricture, intractable vomiting, asthma, chronic bronchitis, and aspiration pneumonia. Pharmacological therapy is available and commonly used. However, this therapy does address the fundamental problem of stomach content flowing in the inappropriate retrograde and into the esophagus.

Normally, the lower esophageal sphincter (LES) allows food to pass from the esophagus to the stomach, while otherwise remaining closed, thus preventing reflux. Closure of the LES is an active process, requiring a combination of proper mechanics and intact innervation. Additionally, the diaphragm may act on the esophagus normally to keep it closed at the LES. Backflow of gastric contents into the esophagus results when gastric pressure is sufficient to overcome the pressure gradient that normally exists at the gastroesophageal junction (GEJ) or when gravity acting on the contents is sufficient to cause flow, retrograde through the GEJ. This situation arises when the gastric pressure is elevated or when the competence of the LES is comprised. Gastric pressure is elevated in association with eating, bending at the waist, squatting, constriction of the waist by clothing, obesity, pregnancy, partial or complete bowel obstruction, etc. Gravitational effects occur when a patient with this condition becomes recumbent. Incompetence of the LES can be functional or anatomic in origin. Function incompetence is associated with hiatus hernia, denervation, myopathy, scleroderma, and chemical or pharmacological influences (smoking, smooth muscle relaxants, caffeine, fatty foods, and peppermint). Anatomic incompetence is associated with congenital malformation, surgical disruption (myotomy, balloon dilatation or bouginage), neoplasm, etc.

The principal types of operations that address the issues with GERD have included some type of reconstruction of the antireflux barrier, which may include a gastric wrap, as in classic Nissen fundoplication, Toupet fundoplication, a nongastric wrap, e.g., the Angelchik prothesis, a ligamentum teres cardiopexy, and fixation of a part of the stomach to an immobile structure, e.g., the preaortic fascia, as in the Hill repair or the anterior rectus sheath. Several of these operations also include a crural repair of the esophageal hiatus in the diaphragm.

Other clinical studies have shown that tightening the LES helps reduce GERD. The requirement is to gather tissue from various locations forming a serosa-to-serosa plication, and securing the tissue position until the tissue unites. The resulting tightening in the LES will increase competency in preventing acid reflux.

Typically, these procedures are performed surgically through an open incision or with traditional laparoscopic and laparotomy techniques. Accordingly, a need exists for methods and devices for approximating tissue using an endoscopic approach.

### SUMMARY OF THE INVENTION

The present invention generally provides devices and methods for approximating tissue. In one embodiment, a device for applying an implantable tissue fastener is provided having an elongate sheath, a handle that can be disposed at a proximal end of the elongate sheath, and a hollow needle that can be disposed within the elongate sheath and be adapted to move independent of the sheath. The elongate sheath can be flexible or it can be rigid. The device can also include a first actuator mechanism that is disposed on the handle and can be operatively associated with the needle such that actuation of the first actuator mechanism is effective to extend the needle from the elongate sheath. A second actuator mechanism can be disposed on the handle and be adapted to deploy a tissue fastener that is disposed within the needle. In one embodiment, the device can further include a tissue backstop that is associated with the hollow needle. A third actuator mechanism can be provided to deploy the tissue backstop. In another embodiment, the device can also include a tissue grasping member that is associated with the elongate sheath and is adapted to engage and manipulate a target region of tissue.

A variety of implantable tissue fasteners are also provided. In one exemplary embodiment, an implantable tissue fastener is provided having an anchor member that includes a continuous body with proximal and distal ends and a longitudinal axis extending therebetween. At least one proximal extension member can be disposed at the proximal end of the anchor member and can extend at an angle with respect to the longitudinal axis of the anchor member. The one-piece fastener can also include at least one distal extension member that is disposed at the distal end of the anchor member and can extend at an angle with respect to the longitudinal axis of the anchor member. The proximal and distal extension members can be configured such that they extend away from the anchor member. In one embodiment, the anchor member can include at least two proximal extension members and at least two distal extension members to form an I-shaped fastener.

In another embodiment, an implantable tissue fastener is provided that can include a first anchor member and a second anchor member. The first anchor member can have a continuous body with a longitudinal axis extending between proximal and distal ends. The proximal end can have at least one extension member extending at an angle with respect to the longitudinal axis of the first anchor member. The second anchor member can be removably matable to the first anchor member and can include at least one extension member extending therefrom. In one embodiment, the first anchor member can have at least two extension members that extend away from the longitudinal axis of the first anchor member to form a substantially T-shaped anchor member. The second anchor member can also have at least two extension members that extend away from the longitudinal axis of the first anchor member to form a substantially C-shaped anchor member.

In yet another embodiment, an implantable tissue fastener is provided that can include first, second, and third anchor members. The first anchor member can have a continuous body with a longitudinal axis extending between proximal and distal ends. The second and third anchor members can be removably matable to the proximal and distal ends of the first anchor member, respectively, and can include at least one extension member extending at an angle with respect to the longitudinal axis of the first anchor member.

In general, the extension members associated with the implantable tissue fasteners can have an arcuate shape. A variety of configurations are available for the terminal ends of the extension members. For example, in one embodiment, the terminal ends can be blunt or rounded. In another embodiment, the terminal ends can be sharp or pointed such that they are adapted to penetrate tissue. The implantable tissue fastener can be formed from a variety of materials. For example, exemplary fasteners can be formed from materials including, but not limited to, stainless steel, titanium, and superelastic alloys such as a nickel titanium alloy. The fastener can be formed entirely from one material or can be formed from any combination of materials.

In another aspect of the invention, a method for approximating tissue is provided and can generally include inserting a device for applying an implantable tissue fastener, positioning the device adjacent a targeted tissue, actuating the device to extend a hollow needle that is disposed within an elongate sheath of the device to penetrate the targeted tissue, reconfiguring the targeted tissue in a desired orientation, and actuating the device to deploy a tissue fastener to penetrate the reconfigured tissue to secure the reconfigured tissue in the desired orientation. In one embodiment, the device can be used in conjunction with an endoscope to facilitate viewing of at least a portion of the method for approximating and can be inserted translumenally through a working channel of the endoscope or through an accessory channel that is mated to the endoscope. In another embodiment, the device can be used in conjunction with a laparoscope and can be inserted through a trocar that extends from an access port. A variety of configurations are available for the approximating device, but the device can generally include an elongate sheath, a hollow needle that is slidably disposed within the sheath, and at least one selectively deployable tissue fastener.

In one embodiment, actuating the device can include partially deploying the tissue fastener so as to engage the targeted tissue with the tissue fastener in a partially deployed configuration. In this embodiment, reconfiguring the targeted tissue can include manipulating the engaged tissue with the partially deployed tissue fastener. In another embodiment, the method can include actuating the device to deploy a tissue backstop disposed on a distal portion of the hollow needle. Reconfiguring the targeted tissue can include compressing the engaged tissue between a proximal surface of the tissue backstop and a distal surface of the elongate sheath. In general, reconfiguring the targeted tissue can include engaging and manipulating an inner surface of the targeted tissue to change the shape of the targeted tissue. The method can further include repeating the steps of reconfiguring the targeted tissue and actuating the device to deploy the tissue fastener.

### BRIEF DESCRIPTION OF THE DRAWINGS

The invention will be more fully understood from the following detailed description taken in conjunction with the accompanying drawings, in which:

FIG. 1 is a perspective view of one embodiment of a device for applying an implantable tissue fastener;

FIG. 2 is a perspective view of the distal end of the device shown in FIG. 1;

FIG. 3A is a perspective view of one embodiment of an implantable tissue fastener;

FIG. 3B is a perspective view of another embodiment of an implantable tissue fastener;

FIG. 3C is a perspective view of another embodiment of an implantable tissue fastener;

FIG. 3D is a perspective view of another embodiment of an implantable tissue fastener;

FIG. 3E is a perspective view of another embodiment of an implantable tissue fastener;

FIG. 4A is a perspective view of one embodiment of an implantable tissue fastener;

FIG. 4B is a perspective view of another embodiment of an implantable tissue fastener;

FIG. 5A is a perspective view of one embodiment of an implantable tissue fastener;

FIG. 5B is a perspective view of another embodiment of an implantable tissue fastener;

FIG. 5C is a perspective view of another embodiment of an implantable tissue fastener;

FIG. 6A is a perspective view of one embodiment of a device for applying an implantable tissue fastener positioned adjacent a target tissue;

FIG. 6B is a perspective view of one embodiment of a device for applying an implantable tissue fastener penetrating a target tissue;

FIG. 6C is a perspective view of one embodiment of a device for applying an implantable tissue fastener penetrated through a target tissue;

FIG. 6D is a perspective view of one embodiment of a device for applying an implantable tissue fastener deploying a tissue fastener to a target tissue;

FIG. 6E is a perspective view of one embodiment of an implantable tissue fastener deployed in a target tissue;

FIG. 7A is a perspective view of another embodiment of a device for applying an implantable tissue fastener penetrating a target tissue;

FIG. 7B is a perspective view of another embodiment of an implantable tissue fastener deployed in a target tissue;

FIG. 8A is a perspective view of another embodiment of a device for applying an implantable tissue fastener penetrating a target tissue;

FIG. 8B is a perspective view of the device shown in FIG. 8A applying an implantable tissue fastener deploying a tissue fastener to a target tissue; and

FIG. 8C is a perspective view of another embodiment of an implantable tissue fastener deployed in a target tissue.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for approximating tissue. The methods and devices utilize a device for applying an implantable tissue fastener and a variety of implantable tissue fasteners. The tissue-fastening device can be delivered endoscopically and can be adapted to function along side of or in conjunction with a flexible endoscope. In general, the device can include a flexible shaft having an implantable tissue fastener applier disposed at a distal end thereof and a handle for operating the implantable tissue fastener applier disposed at a proximal end thereof. For example, in an exemplary embodiment, the device can have a flexible elongate sheath, a handle that is disposed at a proximal end of the elongate sheath, and a hollow needle that is disposed within the elongate sheath and is adapted to move independent of the sheath. At least one actuator mechanism can be disposed on the handle for actuating various operations of the device. For example, a first actuator mechanism can be operatively associated with the needle such that actuation of the first actuator mechanism is effective to extend the needle from the elongate sheath. The device can further include a second actuator mechanism that is adapted to deploy a tissue fastener disposed within the needle. Although the device is shown and described for endoscopic use, one skilled in the art will appreciate that device could include a rigid shaft for laproscopic use.

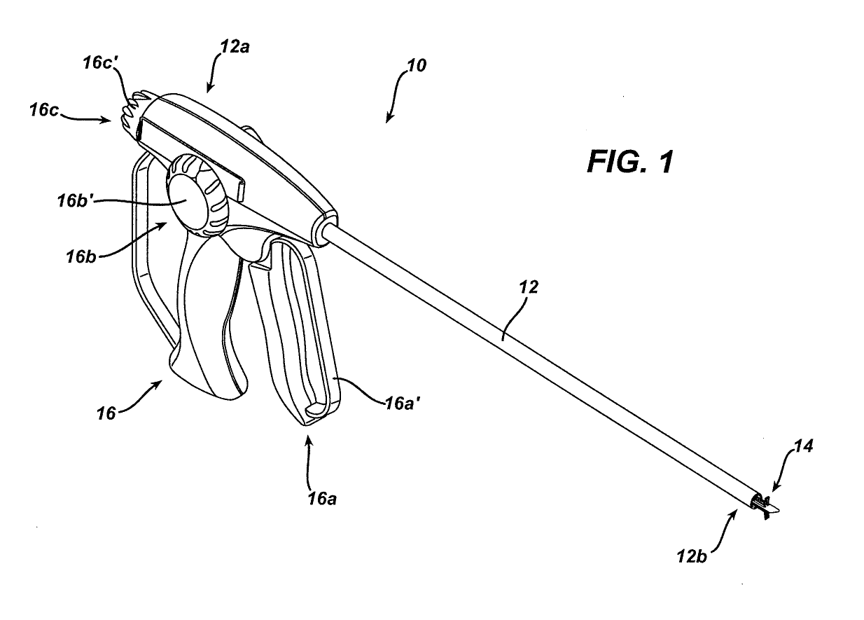

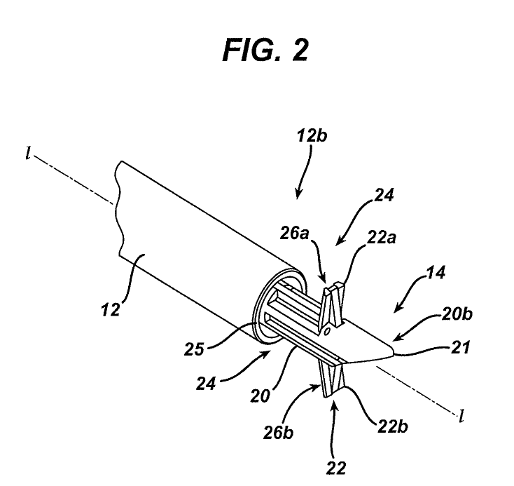

FIGS. 1 and 2 illustrate one exemplary embodiment of a device 10 for applying an implantable tissue fastener. As indicated above, the device can generally include a flexible elongate sheath 12 having an implantable tissue applier 14 disposed at a distal end 12b thereof and a handle 16 for operating the implantable tissue applier 14 at a proximal end 12a thereof. The implantable tissue applier 14 can have a variety of configurations, but in one exemplary embodiment, shown in FIGS. 1 and 2, the tissue applier 14 takes the form of a hollow needle 20 (FIG. 2) that is slidably disposed within the flexible elongate sheath 12. The hollow needle 20 can have a variety of shapes and sizes, but can generally be sized and shaped such that it can slidably move with respect to the sheath 12 and retain an implantable tissue fastener therein. The hollow needle 20 can also be adapted such that it can move independent of the sheath 12.

FIG. 2 shows the hollow needle 20 in a deployed position wherein the hollow needle 20 is advanced distally with respect to the sheath 12 such that the hollow needle 20 extends beyond the distal end 12b of the sheath 12. As illustrated in FIG. 2, the distal end 20b of the hollow needle 20 includes a tissue penetrating tip 21 that can allow the hollow needle 20 to pierce a target region of tissue to approximate. The distal portion 20b of the hollow needle 20 can also be adapted to release or deploy at least one implantable tissue fastener that is disposed therein.

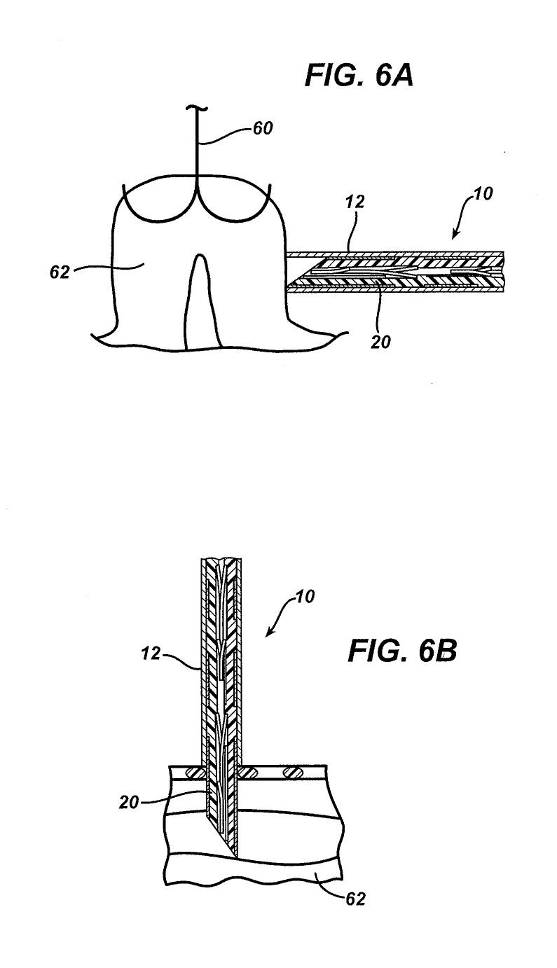

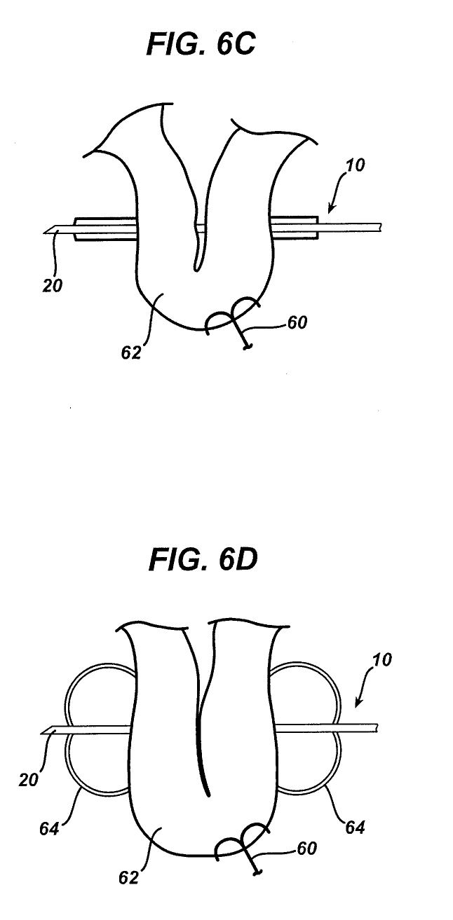

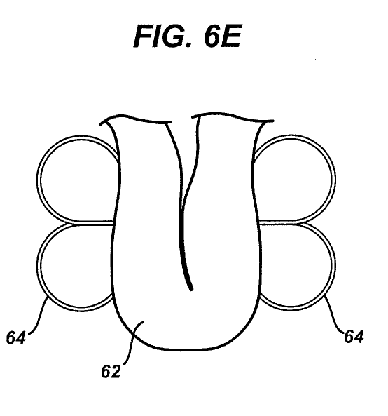

The hollow needle 20 can further include a tissue backstop 22 to aid in isolating and compressing a target tissue. The backstop 22 can have a variety of configurations, but in one exemplary embodiment, shown in FIG. 2, the backstop 22 includes first and second stop members 22a, 22b that are positioned near the distal end 20b of the hollow needle 20. The first and second stop members 22a, 22b can be contained within and/or seated flush with an outer surface 23 of the hollow needle 20 when not in use. For compressing a target tissue, the first and second stop members 22a, 22b can be deployed such that they extend transverse to a longitudinal axis L of the hollow needle 20. FIG. 2 illustrates that such a configuration forms a compression region 24 between a distal facing surface 25 of the elongate sheath 12 and a proximal facing surface 26a, 26b of the first and second stop members 22a, 22b. The device can be adapted to deploy the first and second stop members 22a, 22b simultaneously and/or sequentially depending on the desired use. Although the tissue backstop 22 is shown and described as first and second stop members 22a, 22b, one skilled in the art will appreciate that any combination of stop members can be incorporated into the device 10. Additionally, the device 10 need not have a tissue backstop 22. In such a configuration, the target tissue can be isolated and compressed using a tissue grasping member 60 (shown in FIGS. 6A, 6C-6D, 7A, and 8A-8B) or a partially deployed tissue fastener.

The handle portion 16 of the device can have a variety of configurations but is generally positioned at a proximal end 12a of the elongate sheath 12 and is configured to operate the implantable tissue applier 14 described above. In one exemplary embodiment, the handle 16 can include one or more actuator mechanisms for actuating various operations of the tissue approximating procedure. As shown in FIG. 1, the device includes a first actuator mechanism 16a for moving the hollow needle 20 with respect to the sheath 12, a second actuator mechanism 16b for deploying the tissue backstop 22 associated with the hollow needle 20, and a third actuator mechanism 16c for deploying an implantable tissue fastener that can be disposed within the hollow needle 20. In the embodiment shown, the first actuator mechanism 16a takes the form of a trigger 16a′ that is adapted to advance the hollow needle 20 from the sheath 12 upon compression of the trigger 16a′. The second and third actuator mechanisms 16b, 16c are shown as knobs 16b′, 16c′ that can be rotated to deploy the tissue backstop 22 and tissue fastener, respectively. Although the device 10 is shown and described as having three separate actuator mechanisms 16a, 16b, 16c, one skilled in the art will appreciate that a variety of combinations and configurations of actuator mechanisms can be used to carry out the operations of the tissue approximating procedure.

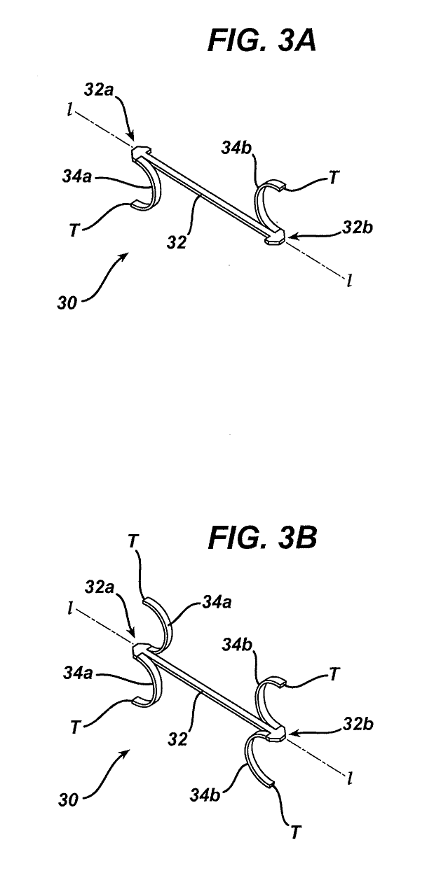

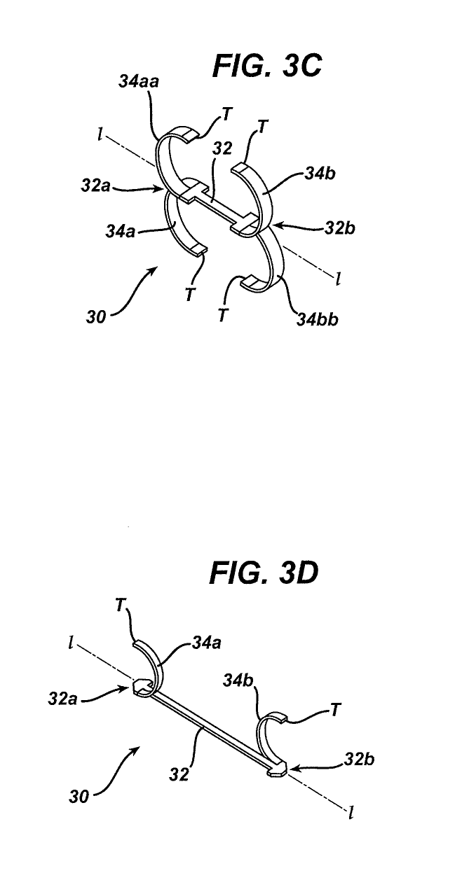

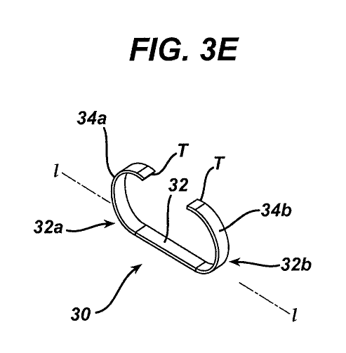

A variety of implantable tissue fasteners can be used with the tissue fastener applier device described above. For example, in one exemplary embodiment, shown in FIGS. 3A-3E, the implantable tissue fastener is in the form of a one-piece fastener 30 that includes an anchor member 32 with at least one proximal and one distal extension member 34a, 34b. The anchor member 32 can have a continuous body with proximal and distal ends 32a, 32b and a longitudinal axis l extending therebetween. At least one proximal extension member 34a can be disposed at the proximal end 32a of the anchor member 32 and can extend at an angle with respect to the longitudinal axis l of the anchor member 32. The fastener 30 can also include at least one distal extension member 34b that is disposed at the distal end 32b of the anchor member 32 and extends at an angle with respect to the longitudinal axis l of the anchor member 32.

The proximal and distal extension members 34a, 34b can extend away from the anchor member 32 to form the one-piece fastener 32. In one embodiment, shown in FIG. 3A, the proximal and distal extension members 34a, 34b extend away from the anchor member 32 on opposite sides of the anchor 32 to form a Z-shaped fastener. In another embodiment, shown in FIGS. 3D-3E, the extension members 34a, 34b extend away from the anchor member 32 on the same side of the anchor 32 to form a U-shaped fastener. The one-piece fastener 30 is not limited to one proximal and one distal extension member, as in some embodiments the anchor member 32 can include at least two proximal extension members 34a, 34aa and/or at least two distal extension members 34b, 34bb. FIGS. 3B-3C illustrate that such a configuration yields an I-shaped fastener. Although the one-piece fastener 30 is shown and described as either a Z, U, or I-shaped fastener, one skilled in the art will appreciate that any number of extension members can be used to form fasteners having a variety of shapes and configurations.

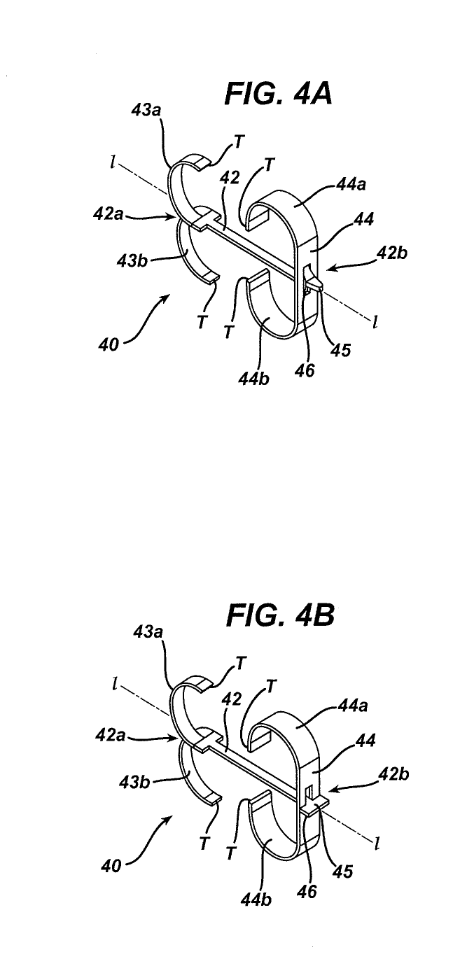

FIGS. 4A-4B illustrate another exemplary embodiment of an implantable tissue fastener wherein the tissue fastener is formed from two separate fastener components. The two-piece tissue fastener 40 can include a first anchor member 42 and a second anchor member 44. As shown in FIGS. 4A-4B, the first anchor member 42 has a continuous body with a longitudinal axis l extending between proximal and distal ends 42a, 42b. The proximal end 42a can have at least one extension member 43a extending at an angle with respect to the longitudinal axis 1 of the first anchor member 42. The second anchor member 44 can be removably matable to the first anchor member 42 and can include at least one extension member 44a extending therefrom. FIGS. 4A-4B illustrate first and second anchor members 42, 44 having first and second extension members 43a, 43b, 44a, 44b extending therefrom. As shown, the first anchor member 42 has at least two extension members 43a, 43b that extend at an angle with respect to the longitudinal axis l of the first anchor member 42 to form a substantially T-shaped anchor member. The second anchor member 44 can also have at least two extension members 44a, 44b that extend at an angle with respect to the longitudinal axis l of the first anchor member 42 to form a substantially C-shaped anchor member. The distal end 42b of the first anchor member 42 can include a mating element 45 that is formed thereon and is adapted to mate to a complementary mating element 46 disposed on the second anchor member 44. The mating elements can have a variety of configurations. For example, in an exemplary embodiment, shown in FIGS. 4A-4B, the mating element 45 disposed on the distal end 42b of the first anchor member 42 is a blunt tab that is adapted to slidably engage an opening formed in the second anchor member 44. As shown in FIG. 4B, the opening is sized and shaped such that blunt tab can achieve a snap fit when received by the second anchor member 44. Other exemplary embodiments for the mating elements include, but are not limited to, threaded fasteners, twist locks, and interference fits.

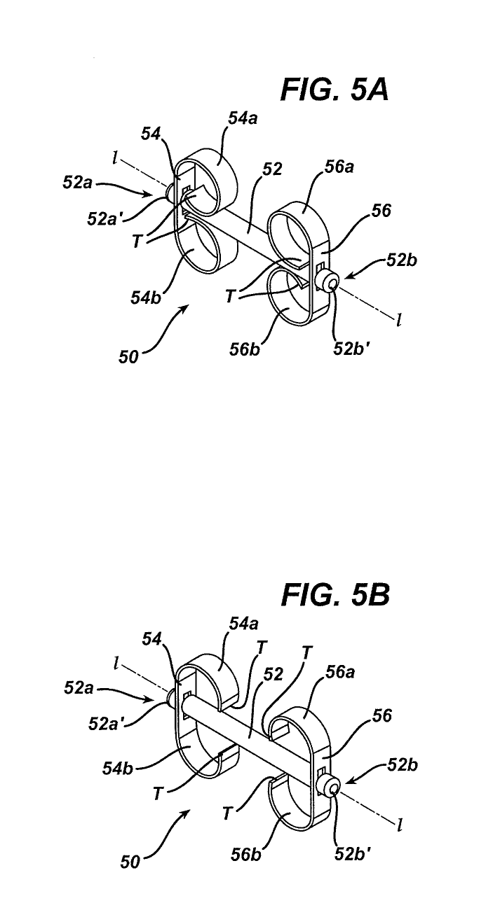

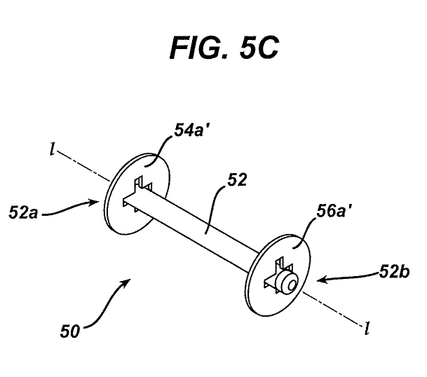

FIGS. 5A-5C illustrate another exemplary embodiment of an implantable tissue fastener wherein the tissue fastener is formed from three separate fastener components. The three-piece tissue fastener 50 can include first, second, and third anchor members 52, 54, 56. The first anchor member 52 can have a continuous body with a longitudinal axis l extending between proximal and distal ends 52a, 52b. As shown in FIGS. 5A-5C, the first anchor member 52 is an elongate member with mating elements 52a′, 52b′ disposed on the proximal and distal ends 52a, 52b. The second and third anchor members 54, 56 can be removably matable to the proximal and distal ends 52a, 52b of the first anchor member 52, respectively. In one exemplary embodiment, shown in FIGS. 5A-5B, the second and third anchor members 54, 56 include at least one extension member 54a, 54b, 56a, 56b extending at an angle with respect to the longitudinal axis l of the first anchor member 52. In another embodiment, shown in FIG. 5C, the extension members 54a′, 56a′ are disks that can be mated to the proximal and distal ends 52a, 52b of the first anchor member 52 so as to form a barbell type fastener when assembled. The second and third extension members 54, 56 can be mated to the first anchor member 52 using any of the techniques described above with reference to the two-piece fastener 40.

In general, the extension members associated with the implantable tissue fasteners 30, 40, 50 can have an arcuate shape. When deployed, the arcuate shape can provide a rounded surface for contacting and applying a fastening load to the tissue. The length of the extension member can vary such that the arcuate extension member can be substantially circular (FIG. 5A) or semi-circular (FIGS. 3A-3E, 4A-4B, and 5B) in shape. The orientation of the arcuate extension members can also vary. In one exemplary embodiment, shown in FIGS. 3C, 3E, 4A-4B, and 5A-5B, the arcuate extension member is oriented such that the terminal ends T of the members curve inward or toward each other. In another exemplary embodiment, shown in FIGS. 3A-3B and 3D, the arcuate shape is oriented such that the terminal ends T curve outward or away from each other. A variety of configurations are available for the terminal ends of the extension members. For example, in one embodiment, the terminal ends can be blunt or rounded, as it is not necessary for the terminal ends to penetrate tissue. Alternatively, the terminal ends can be sharp or pointed such that they are adapted to penetrate tissue.

The implantable tissue fastener can be formed from a variety of materials. For example, exemplary fasteners can be formed from materials including, but not limited to, stainless steel, titanium, and superelastic alloys such as a nickel titanium alloy. The fastener can be formed entirely from one material or can be formed from any combination of materials. For example, in an exemplary embodiment, the anchor member can be formed from stainless steel and the proximal and distal extension members can be formed from a superelastic alloy. Such a configuration can allow the fastener to be retained within the hollow needle in a constrained position and revert to its unconstrained shape upon deployment or release from the needle. The materials can also be selected such that the fastener can be used as a marker when deployed in a target tissue.

The present invention also provides methods of approximating a target region of tissue. The method can include inserting a device for applying an implantable tissue fastener translumenally. The device can be used in conjunction with an endoscope to facilitate viewing of at least a portion of the method for approximating and can be inserted through a working channel of the endoscope or through an accessory channel that is mated to the endoscope. The approximating device can take the form of any of the embodiments described above but can generally include an elongate sheath, a hollow needle that is slidably disposed within the sheath, and at least one selectively deployable tissue fastener. FIGS. 6A-6E illustrate one exemplary embodiment of approximating a target region of tissue. As shown in FIG. 6A, the approximating device 10 is positioned adjacent the target tissue 62 of an internal organ such as the stomach. Once the device 10 is positioned adjacent the target tissue 62, the device 10 can be actuated to extend the hollow needle 20 from the elongate sheath 12 and penetrate the targeted tissue 62 as is shown in FIGS. 6B-6C.

The target tissue can then be reconfigured in a desired orientation. Reconfiguring the targeted tissue can include engaging and manipulating an inner surface of the targeted tissue to change the shape of the targeted tissue. The reconfigured tissue can take a variety of shapes including, for example, a fold, a bulge, a mound, a plication, a ridge, a tube, a cone, and a horn. FIGS. 6A-6C show the target tissue 62 being reconfigured or manipulated by a separate tissue grasping member 60 or retractor; however, the method need not include such a step. In one exemplary embodiment, the approximating device 10 can include a tissue backstop for manipulating the target tissue. In such an embodiment, once the device is actuated to penetrate the targeted tissue, the device can be actuated to deploy the tissue backstop. The target tissue can then be manipulated by compressing the tissue between a proximal facing surface of the tissue backstop and a distal facing surface of the sheath.

After manipulating the tissue to reconfigure the tissue in a desired orientation, the device 10 can be actuated to deploy a tissue fastener 64 to the target tissue to penetrate the reconfigured tissue to secure the tissue in the reconfigured orientation. In one embodiment, shown in FIG. 6D, actuating the device 10 is effective to fully deploy the fastener 64. For example, fastener 64 can be retained within the hollow needle 20 in a constrained position such that upon deployment or release from the needle the fastener will revert to its unconstrained shape to engage and secure the reconfigured tissue. In another embodiment, actuating the device can be effective to partially deploy the tissue fastener so as to engage the targeted tissue with the tissue fastener in a partially deployed configuration. In this embodiment, the target tissue can be manipulated and reconfigured using the partially deployed fastener. Once the desired orientation is achieved, the device can again be actuated to fully deploy the partially deployed fastener and secure the tissue in the reconfigured orientation.

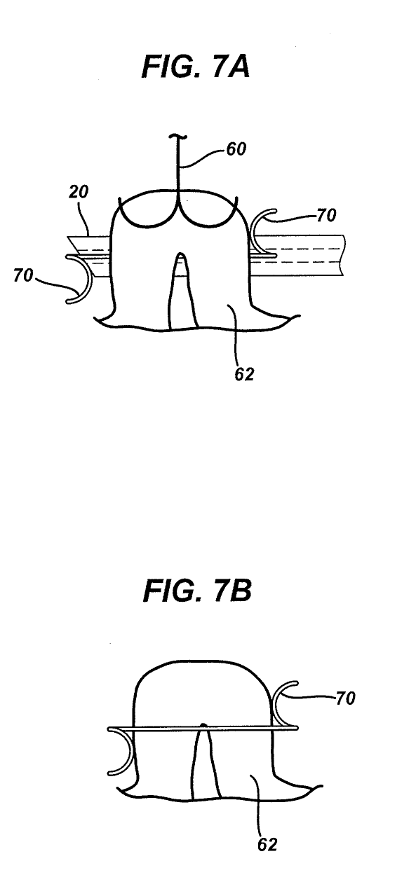

The steps of reconfiguring the target tissue and actuating the device to deploy a tissue fastener can be repeated as needed. Once the target tissue is reconfigured and secured as desired, the needle can be retracted within the sheath and the device can be removed from the treatment site. FIG. 6E shows an exemplary embodiment of a reconfigured target tissue 62 secured by a fastener 64 following the removal of the device 10. A fastener of the type shown in FIGS. 3A-3E is illustrated in FIGS. 7A-7B which demonstrate the deployment of a one-piece, Z-shaped fastener 70.

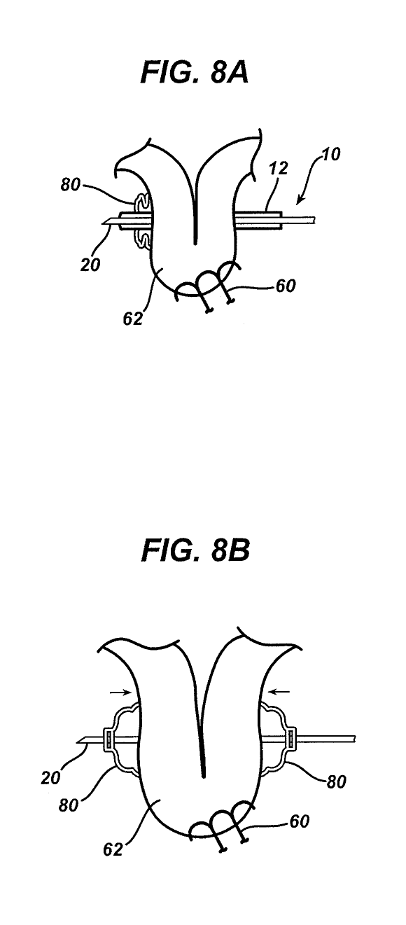

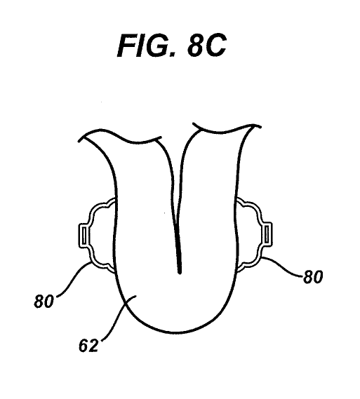

FIGS. 8A-8C illustrate the deployment of a three-piece barbell type fastener 80. FIG. 8A shows the barbell fastener being partially deployed and used to manipulate and reconfigure the target tissue. A variety of techniques can be used to implant a multi-piece fastener. For example, in one exemplary embodiment the first, second, and third anchor members can all be contained within the hollow needle and released individually to engage the target tissue. Such a configuration can allow all or some of the anchor members to be used to manipulate and reconfigure the target tissue. In one embodiment, the first and second anchor members can be deployed and used to reconfigure the tissue. Once the tissue is reconfigured as desired, the third anchor member can be deployed from the hollow needle and can be mated to the first and second anchor members to secure the reconfigured tissue. Although the method is described above as delivering an entire multi-piece fastener via the approximating device 10, one skilled in the art will appreciate that the hollow needle 20 need not carry each anchor member of a multi-piece fastener, as a separate device can be used to deliver one or more anchor members if desired.

A person skilled in the art will appreciate that the present invention has application in conventional endoscopic and open surgical instrumentation as well application in robotic-assisted surgery.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for approximating tissue, comprising:
inserting to a target site a device for applying an implantable tissue fastener, the device having an elongate sheath, a hollow needle slidably disposed within the sheath, and at least one selectively deployable tissue fastener;
positioning the device adjacent the targeted tissue;
actuating the device to extend the hollow needle from the elongate sheath and penetrate the targeted tissue;
reconfiguring the targeted tissue in a desired orientation; and
actuating the device to deploy the tissue fastener to penetrate the reconfigured tissue to secure the reconfigured tissue in the desired orientation.

2. The method of claim 1, wherein actuating the device includes partially deploying the tissue fastener so as to engage the targeted tissue with the tissue fastener in a partially deployed configuration.

3. The method of claim 2, wherein reconfiguring the targeted tissue comprises manipulating the engaged tissue with the partially deployed tissue fastener.

4. The method of claim 1, further comprising actuating the device to deploy a tissue backstop disposed on a distal portion of the hollow needle.

5. The method of claim 4, wherein reconfiguring the targeted tissue comprises compressing the engaged tissue between a proximal surface of the tissue backstop and a distal surface of the elongate sheath.

6. The method of claim 2, further comprising actuating the device to fully deploy the tissue fastener.

7. The method of claim 1, further comprising repeating the steps of reconfiguring the targeted tissue and actuating the device to deploy the tissue fastener.

8. The method of claim 1, wherein reconfiguring the targeted tissue comprises engaging and manipulating an inner surface of the targeted tissue to change the shape of the targeted tissue.

9. The method of claim 8, wherein the targeted tissue is reconfigured to be in the shape selected from the group consisting of a fold, a bulge, a mound, a plication, a ridge, a tube, a cone, and a horn.

10. The method of claim 1, wherein reconfiguring and approximating the targeted tissue are performed using only the tissue fastener delivery device.

11. The method of claim 1, wherein inserting the device comprises inserting the device translumenally.

12. The method of claim 1, wherein inserting the device comprises inserting the device laparoscopically.

13. The method of claim 1, further comprising endoscopically viewing at least a portion of the method for approximating.

14. The method of claim 1, wherein the tissue fastener is a one piece fastener.

15. The method of claim 1, wherein the tissue fastener comprises first and second anchor members.

16. The method of claim 1, wherein the tissue fastener comprises first, second, and third anchor members.

17. The method of claim 1, wherein inserting the device for applying an implantable tissue fastener comprises inserting the device through a working channel of an endoscope.

18. The method of claim 1, wherein inserting the device for applying an implantable tissue fastener comprises inserting the device through an accessory channel mated to an endoscope.

19. The method of claim 1, wherein the targeted tissue is an internal organ that is endscopically accessed.
